(19) United States
(12) Patent Application Publication
(10) Pub. No.: US 2005/0202049 A1
(43) Pub. Date: Sep. 15, 2005

(54) Retinoid-containing preparations

(75) Inventors: Axel Jentzsch, Mannheim (DE); Andreas Habich, Speyer (DE); Christian Kopsel, Weinheim (DE); Andreas Ernst, Worms (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/515,636

(22) PCT Filed: Jul. 16, 2003
(86) PCT No.: PCT/EP03/07700

§ 371 Date: Dec. 7, 2004

(30) Foreign Application Priority Data
Jul. 24, 2002 (DE) 102 337 40.3

(51) Int. Cl. A61K 9/00; A61K 47/00; A61K 31/355; A61K 31/07

(52) U.S. Cl. 424/400; 514/725; 514/458; 514/474; 424/439

(57) ABSTRACT

Preparations comprising at least one retinoid, at least one water-soluble antioxidant and at least one oil-soluble antioxidant, where, per part by weight of retinoid, at least 1 part by weight of one or more water-soluble antioxidants and 0.1 to 100 parts by weight of one or more oil-soluble antioxidants are present in the preparations, where the content of one or more water-soluble antioxidants is in the range from 0.05 to 0.8% by weight, based on the total amount of the preparations.

The present invention relates to retinoid-containing preparations, their production and use in cosmetics, pharmacy, and also in the food sector.

Retinoids are some of the most active ingredients which are used in cosmetics and in dermatology. They have, inter alia, a regulating effect on normal cell growth and influence the differentiation of epithelial cells. Retinoic acids are thus used for the treatment of acne and retinol is used in antiwrinkle creams.

However, the use of retinoids is severely restricted, which can be attributed, inter alia, to the high instability of the compounds. For this reason, strict safety precautions have to be observed during the production of retinoid-containing preparations. For example, the production must take place entirely under protective gas and the finished product must be packaged in oxygen-impermeable packaging.

These requirements demand that the manufacturer has complex technological equipment and are thus associated with high production costs.

A number of methods of stabilizing retinoids have been described. For example, EP-A-1 055 720 discloses the stabilization of oxygen-sensitive compounds by using thio compounds or glycoproteins with the exclusion of oxygen.

According to WO 93/00085 and EP-A-0 440 398, both water- and also fat-soluble antioxidants are used together with chelate-forming agents for the stabilization of retinoids.

However, the methods described do not always lead to adequate stabilization for the purposes of the invention. Instead, both water-soluble antioxidants, in particular ascorbic acid, and also fat-soluble antioxidants, in particular tocopherol, display a destabilizing effect in connection with retinoids in certain concentration ranges.

In addition, under some circumstances, the use of certain antioxidant combinations together with retinoids may lead to undesired secondary effects, e.g. yellowish discolorations of the preparations, which render the use of these systems in cosmetics or in the food sector unfeasible.

It is an object of the present invention to provide retinoid-containing preparations which do not have the disadvantages given above with regard to stability and discoloration, and which can be produced in a simple manner.

We have found that this object is achieved by preparations comprising at least one retinoid, at least one water-soluble antioxidant and at least one oil-soluble antioxidant, where, per part by weight of retinoid, at least 1 part by weight of one or more water-soluble antioxidants and 0.1 to 100 parts by weight of one or more oil-soluble antioxidants are present in the preparations, where the content of one or more water-soluble antioxidants is in the range from 0.05 to 0.8% by weight, based on the total amount of the preparations.

The preparations according to the invention are cosmetic and dermatological or pharmaceutical preparations, and preparations for the food sector. Preference is given to cosmetic preparations, in particular skincare preparations.

An advantageous embodiment of the preparations according to the invention comprises, per part by weight of retinoid, 1 to 100 parts by weight, preferably 1 to 20 parts by weight, particularly preferably 1 to 5 parts by weight, very particularly preferably 2 to 3 parts by weight, of one or more water-soluble antioxidants and 1 to 20 parts by weight, preferably 3 to 10 parts by weight, particularly preferably 5 to 10 parts by weight, very particularly preferably 8 to 10 parts by weight, of one or more oil-soluble antioxidants.

For the purposes of the present invention, retinoids means vitamin A alcohol (retinol) and its derivatives, such as vitamin A aldehyde (retinal), vitamin A acid (retinoic acid) and vitamin A esters, such as retinyl acetate and retinyl palmitate. The term retinoic acid here includes both all-trans retinoic acid and also 13-cis retinoic acid. The terms retinol and retinal preferably include the all-trans compounds. The preferred retinoid used for the preparations according to the invention is all-trans-retinol.

The water-soluble antioxidants intended are, inter alia, ascorbic acid, sodium sulfite, sodium metabisulfite, sodium bisulfite, sodium thiosulfite, sodium formaldehyde sulfoxylate, isoascorbic acid, thioglycerol, thiosorbitol, thiourea, thioglycolic acid, cysteine hydrochloride, 1,4-diazobicyclo(2.2.2)octane or mixtures thereof.

Preferred water-soluble antioxidants are ascorbic acid (L-ascorbic acid) and isoascorbic acid (D-ascorbic acid), particularly preferably L-ascorbic acid.

The L-ascorbic acid which is particularly preferably used may be the free acid or else salts thereof. Examples of salts of L-ascorbic acid are alkali metal or alkaline earth metal salts of L-ascorbic acid, such as sodium L-ascorbate, potassium L-ascorbate or calcium L-ascorbate, but also salts of L-ascorbic acid with an organic amine compound, such as choline ascorbate or L-carnitine ascorbate. Very particular preference is given to using the free L-ascorbic acid or sodium L-ascorbate. Corresponding statements apply to the use of D-ascorbic acid.

The oil-soluble antioxidants intended are, inter alia, butylated hydroxytoluene (BHT), ascorbyl palmitate, butylated hydroxyanisole, α-tocopherol, phenyl-α-naphthylamine or mixtures thereof.

A preferred oil-soluble antioxidant is α-tocopherol, which may either be (R,R,R)- or (all-rac)-α-tocopherol.

The preparations according to the invention usually comprise 0.005 to 0.5% by weight, preferably 0.01 to 0.5% by weight, particularly preferably 0.03 to 0.15% by weight, very particularly preferably 0.04 to 0.12% by weight, of one or more retinoids, in particular all-trans-retinol, 0.05 to 0.8% by weight, preferably 0.1 to 0.5% by weight, particularly preferably 0.15 to 0.3% by weight, of one or more water-soluble antioxidants, in particular L-ascorbic acid, 0.0005 to 2% by weight, preferably 0.01 to 1.8% by weight, particularly preferably 0.1 to 1.5% by weight, very particularly preferably 0.4 to 1.2% by weight, of one or more oil-soluble antioxidants, in particular α-tocopherol.

The preparations according to the invention are characterized inter alia in that it is possible to dispense with the use of protective gas during their production, bottling and storage while simultaneously ensuring adequate stability.

For the purposes of the invention, adequate stability is understood as meaning that the retinoid is recovered in an amount of at least 90% in the preparation after storage for 12 weeks at 40° C. In addition, no undesired color changes arise during storage of the preparations according to the invention.

It is advantageous if the preparations according to the invention are stored in oxygen-impermeable packagings.

The oxygen-impermeable packagings may be any standard commercial packagings suitable for this purpose, such as, for example, glass containers or aluminum packagings. Preference is given to using aluminum tubes, particular preference is given to those aluminum tubes whose inside has additionally been provided with a protective coating.

The cosmetic and the dermatological or pharmaceutical preparations are generally based on a carrier which comprises at least one oil phase. However, preparations merely based on water are also possible. Accordingly, oils, creams, pastes or non-greasy gels or, preferably, emulsions, are suitable.

Suitable emulsion are O/W emulsions, W/0 emulsions, microemulsions or multiple emulsions, such as O/W/O emulsions or W/O/W emulsions with one or more retinoids according to the invention present in dispersed form, where the emulsions are obtainable, for example, by phase inversion technology, as in DE-A-197 26 121.

Customary cosmetic auxiliaries which may be suitable as additives for the cosmetic or pharmaceutical preparations are, for example, coemulsifiers, fats and waxes, stabilizers, thickeners, biogenic active ingredients, film formers, fragrances, dyes, pearlizing agents, preservatives, pigments, electrolytes (e.g. magnesium sulfate) and pH regulators. Suitable coemulsifiers are preferably known W/O and also O/W emulsifiers, such as, for example, polyglycerol esters, sorbitan esters or partially esterified glycerides. Typical examples of fats are glycerides; waxes which can be mentioned are, inter alia, beeswax, paraffin wax or microcrystalline waxes, optionally in combination with hydrophilic waxes. Stabilizers which may be used are metal salts of fatty acids, such as, for example, magnesium stearate, aluminum stearate and/or zinc stearate. Suitable thickeners are, for example, crosslinked polyacrylic acids and derivatives thereof, polysaccharides, in particular xanthan gum, guar guar, agar agar, alginates and Tyloses, carboxymethylcellulose and hydroxyethylcellulose, and also fatty alcohols, monoglycerides and fatty acids, polycrylates, polyvinyl alcohol and polyvinylpyrrolidone. Biogenic active ingredients are understood as meaning, for example, plant extracts, protein hydrolyzates and vitamin complexes. Customary film formers are, for example, hydrocolloids, such as chitosan, microcrystaline chitosan or quaternized chitosan, polyvinylpyrrolidone, vinylpyrrolidone-vinyl acetate copolymers, polymers of the acrylic acid series, quaternary cellulose derivatives and similar compounds. Suitable preservatives are, for example, formaldehyde solution, p-hydroxybenzoate or sorbic acid. Suitable pearlizing agents are, for example, glycol distearic esters, such as ethylene glycol distearate, but also fatty acids and fatty acid monoglycol esters. Dyes which can be used are the substances approved and suitable for cosmetic purposes, as listed, for example, in the publication “Kosmetische Färbemittel” [Cosmetic Colorants] from the Farbstoffkommission der Deutschen Forschungsgemeinschaft [Dyes Commission of the German Research Society], published by Verlag Chemie, Weinheim, 1984. These dyes are customarily used in a concentration of from 0.001 to 0.1% by weight, based on the total mixture.

The use of further antioxidants is advantageous in many cases. Thus, in addition to the antioxidants according to the invention mentioned at the outset, it is possible to use all antioxidants customary or suitable for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D, L-carnosine, D-carnosine, L-carnosine and derivatives thereof (e.g. anserine), carotenoids, carotenes (e.g. β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thiorodoxin, glutathione, cystine, cystamine and their glycosyl, N-acetyl, methyl, ethyl, propyl, amyl, butyl and lauryl, palmitoyl, oleyl, γ-linoleyl, cholesteryl and glyceryl esters) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthioninesulfoximines, homocysteinesulfoximines, buthioninesulfones, penta-, hexa-, heptathioninesulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, biliburin, biliverdin, EDTA and derivatives thereof, unsaturated fatty acids and derivatives thereof (e.g. γ-linolenic acid, linoleic acid, oleic acid), folic acid and derivatives thereof, and coniferyl benzoate of benzoin resin, rutinic acid and derivatives thereof, α-glycosylrutin, ferulic acid, furfurylideneglucitol, carnosine, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide).

The total amount of the abovementioned antioxidants (one or more compounds) in the preparations is preferably 0.075 to 30% by weight, particularly preferably 0.1 to 20% by weight, in particular 0.55 to 10% by weight, based on the total weight of the preparation.

Customary oil components in cosmetics are, for example, paraffin oil, glyceryl stearate, isopropyl myristate, diisopropyl adipate, cetylstearyl 2-ethylhexanoate, hydrogenated polyisobutene, vaseline, caprylic/capric triglycerides, microcrystalline wax, lanolin and stearic acid.

The preparations according to the invention are suitable, inter alia, as additive for foods, for example for vitaminizing drinks and for the production of food supplement preparations in the human and animal sector.

For the use of the retinoid-containing preparations according to the invention in foods, all of the abovementioned types of formulation are suitable in principle. Thus, for the vitaminization of drinks, it is possible, for example, to use emulsions, solubilizates or else water-dispersible dry powders in which mixtures of at least one retinoid, at least one water-soluble antioxidant and at least one oil-soluble antioxidant are present.

It is, however, also possible to add emulsions which contain the retinoid-containing preparations according to the invention, milk products, such as yoghurt, flavored milk drinks or dairy ice cream.

The invention also provides food supplements, animal feeds, foods and pharmaceutical and cosmetic preparations comprising the retinoid-containing preparations according to the invention.

Food supplement preparations and pharmaceutical preparations which comprise the retinoid mixture according to the invention are understood as meaning, inter alia, tablets, dragees and hard and soft gelatin capsules.

Production Method:

There are numerous ways of producing a cosmetic preparation. For example, use is made of the hot/hot method, the hot/cold method or the cold/cold method, as described, for example, in “Kosmetik—Entwicklung, Herstellung and Anwendung kosmetischer Mittel” [Cosmetics—Development, production and use of cosmetic compositions], Ed. Wilfried Umbach, Thieme Verlag, 1995, page 511. With the help of these methods it is possible to prepare oil-in-water (O/W), water-in-oil (W/O), but also multiple emulsions and creamgels and gels. The active ingredients are incorporated here preferably after the formulation has been cooled to below 40° C., in particularly sensitive cases, preferably after it has been cooled to room temperature. For the investigations which form the basis of this invention, an O/W emulsion was prepared in the hot/hot method and the active ingredients were then incorporated into the finished formulation at room temperature.

The formulation used:

| | % | Ingredient | INCI |
|---|---|---|---|
| Phase A | 2.00 | Cremophor A 6 | Ceteareth-6, Stearyl |
| | | | Alcohol |
| | 2.00 | Cremophor A 25 | Ceteareth-25 |
| | 3.00 | Jojoba oil | Simmondsia Chinensis |
| | | | (Jojoba) Seed oil |
| | 3.00 | Cetylstearyl alcohol | Cetearyl Alcohol |
| | 10.00 | Paraffin oil, | Mineral Oil |
| | | viscous | |
| | 5.00 | Vaseline | Petrolatum |
| | 4.00 | Miglyol 812 | Caprylic/Capric Triglyceride |
| | 0.10 | BHT | BHT |
| Phase B | 5.00 | 1,2-Propylene glycol | Propylene Glycol |
| | | USP | |
| | 0.10 | Edeta BD | Disodium EDTA |
| | 20.00 | Carbopol 934, | Carbomer |
| | | 1% in water dem. | |
| | 0.30 | Chemag 2000 | |
| | ad 100 | Water dem. | Aqua dem. |
| Phase C | 0.80 | Sodium hydroxide, | Sodium Hydroxide |
| | | 10% in water dem. | |
| Phase D | 0.50 | Vitamin E acetate | Tocopheryl Acetate |
| | 0.20 | Phenoxyethanol | Phenoxyethanol |
| | q.s. | Perfume oil | |

Production:

Phases A and B were heated separately to about 80° C. Phase B was then stirred into phase A and homogenized. Phase C was used to neutralize and then homogenize. With stirring, the cream was cooled to about 40° C., phase D was stirred in and the mixture was homogenized again.

The water-soluble and the oil-soluble antioxidants were then, after cooling the cream to room temperature, incorporated into the finished emulsion. For this, D,L-alpha-tocopherol was firstly added, then the ascorbic acid and then retinol (retinol 15D®, BASF; 15% strength solution of retinol in a medium-chain triglyceride) were introduced with stirring.

The cream was then transferred to aluminum tubes with an internal protective coating.

In accordance with the production method described above, creams containing varying amounts of retinol (all-rac)-α-tocopherol and L-ascorbic acid were produced and stored for 12 weeks at 40° C. for stability investigations. The result of this stability test is given in the table below:

TABLE

| | Retinol | Tocopherol | Ascorbic acid | Base value | 3 weeks | 6 weeks | 9 weeks | 12 weeks |
|---|---|---|---|---|---|---|---|---|
| | ppm | ppm | ppm | % | % | % | % | % |
| 1 | 1000 | 0 | 0 | 100.0 | 87.8 | 79.2 | 73.2 | 67.3 |
| 2 | 1000 | 100 | 10 | 100.0 | 90.6 | 83.2 | 75.7 | 69.7 |
| 3 | 1000 | 100 | 100 | 100.0 | 88.9 | 80.4 | 74.0 | 70.5 |
| 4 | 1000 | 100 | 500 | 100.0 | 89.3 | 82.9 | 76.9 | 76.5 |
| 5 | 1000 | 100 | 1000 | 100.0 | 97.4 | 94.8 | 94.2 | 92.9 |
| 6 | 1000 | 100 | 2000 | 100.0 | 96.6 | 95.5 | 95.2 | 93.4 |
| 7 | 1000 | 100 | 3000 | 100.0 | 97.3 | 95.9 | 95.2 | 93.6 |
| 8 | 1000 | 100 | 5000 | 100.0 | 94.1 | 92.7 | 91.6 | 90.0 |
| 9 | 1000 | 100 | 10000 | 100.0 | 84.8 | 73.0 | 66.8 | 59.4 |
| 10 | 1000 | 100 | 30000 | 100.0 | 60.1 | 39.2 | 24.1 | 16.4 |
| 11 | 1000 | 1000 | 0 | 100.0 | 86.5 | 74.6 | 67.2 | 60.0 |
| 12 | 1000 | 1000 | 10 | 100.0 | 88.1 | 78.7 | 71.5 | 65.9 |
| 13 | 1000 | 1000 | 100 | 100.0 | 88.3 | 77.3 | 70.9 | 60.8 |
| 14 | 1000 | 1000 | 500 | 100.0 | 91.2 | 87.6 | 87.1 | 88.1 |
| 15 | 1000 | 1000 | 1000 | 100.0 | 95.8 | 92.8 | 92.4 | 92.0 |
| 16 | 1000 | 1000 | 2000 | 100.0 | 95.3 | 92.1 | 91.9 | 91.0 |
| 17 | 1000 | 1000 | 3000 | 100.0 | 95.0 | 93.5 | 91.9 | 91.1 |
| 18 | 1000 | 1000 | 5000 | 100.0 | 97.2 | 94.4 | 93.3 | 92.7 |
| 19 | 1000 | 1000 | 10000 | 100.0 | 86.9 | 75.5 | 68.1 | 58.8 |
| 20 | 1000 | 1000 | 30000 | 100.0 | 60.2 | 35.6 | 22.1 | 19.4 |
| 21 | 1000 | 2000 | 0 | 100.0 | 82.0 | 68.9 | 60.0 | 51.3 |
| 22 | 1000 | 2000 | 10 | 100.0 | 87.8 | 75.9 | 66.9 | 58.4 |
| 23 | 1000 | 2000 | 100 | 100.0 | 85.8 | 74.3 | 65.1 | 56.9 |
| 24 | 1000 | 2000 | 500 | 100.0 | 88.9 | 86.9 | 87.3 | 88.9 |
| 25 | 1000 | 2000 | 1000 | 100.0 | 94.7 | 93.3 | 93.4 | 92.2 |
| 26 | 1000 | 2000 | 2000 | 100.0 | 95.6 | 94.2 | 93.8 | 92.7 |
| 27 | 1000 | 2000 | 3000 | 100.0 | 95.6 | 93.7 | 92.9 | 92.3 |
| 28 | 1000 | 2000 | 5000 | 100.0 | 99.3 | 93.9 | 96.2 | 91.6 |
| 29 | 1000 | 2000 | 10000 | 100.0 | 87.3 | 76.2 | 69.8 | 61.5 |
| 30 | 1000 | 2000 | 30000 | 100.0 | 59.9 | 35.1 | 20.0 | 13.2 |
| 31 | 1000 | 3000 | 0 | 100.0 | 77.5 | 63.8 | 52.7 | 46.3 |
| 32 | 1000 | 3000 | 10 | 100.0 | 85.5 | 72.0 | 64.3 | 56.9 |
| 33 | 1000 | 3000 | 100 | 100.0 | 83.6 | 68.9 | 62.2 | 53.2 |
| 34 | 1000 | 3000 | 500 | 100.0 | 92.7 | 88.9 | 97.6 | 88.3 |
| 35 | 1000 | 3000 | 1000 | 100.0 | 95.0 | 92.8 | 91.3 | 91.9 |
| 36 | 1000 | 3000 | 2000 | 100.0 | 96.0 | 94.5 | 94.0 | 93.1 |
| 37 | 1000 | 3000 | 3000 | 100.0 | 95.7 | 93.1 | 91.7 | 91.8 |
| 38 | 1000 | 3000 | 5000 | 100.0 | 98.0 | 93.8 | 93.5 | 92.0 |
| 39 | 1000 | 3000 | 10000 | 100.0 | 88.1 | 74.2 | 69.2 | 60.4 |
| 40 | 1000 | 3000 | 30000 | 100.0 | 60.1 | 35.5 | 21.8 | 13.2 |
| 41 | 1000 | 10000 | 10 | 100.0 | 63.4 | 44.4 | 34.0 | 26.7 |
| 42 | 1000 | 10000 | 100 | 100.0 | 63.7 | 45.5 | 35.6 | 28.0 |
| 43 | 1000 | 10000 | 500 | 100.0 | 71.4 | 68.4 | 68.8 | 66.7 |
| 44 | 1000 | 10000 | 1000 | 100.0 | 101.8 | 95.7 | 95.7 | 95.7 |
| 45 | 1000 | 10000 | 2000 | 100.0 | 102.1 | 97.4 | 96.7 | 97.2 |
| 46 | 1000 | 10000 | 3000 | 100.0 | 102.0 | 96.7 | 93.9 | 96.5 |
| 47 | 1000 | 10000 | 5000 | 100.0 | 99.3 | 93.2 | 91.1 | 90.7 |
| 48 | 1000 | 10000 | 10000 | 100.0 | 88.2 | 76.1 | 69.0 | 60.0 |
| 49 | 1000 | 10000 | 30000 | 100.0 | 60.3 | 38.4 | 21.5 | 14.4 |

The subject-matter of the invention is illustrated in more detail by reference to the formulation examples below.

### EXAMPLE 1

Skin lotion (O/W emulsion)

| | % w/w |
|---|---|
| Ceteareth-6 and stearyl alcohol | 2.50 |
| Ceteareth-25 | 2.50 |
| Hydrogenated cocoglyceride | 1.50 |
| PEG-40 dodecyl glycol copolymer | 3.00 |
| Dimethicone | 3.00 |
| Phenethyl dimethicone | 2.00 |
| Cyclomethicone | 1.00 |
| Cetearyl octanoate | 5.00 |
| Avocado oil | 1.00 |
| Almond oil | 2.00 |
| Wheatgerm oil | 0.80 |
| Panthenol USP | 1.00 |
| Phytantriol | 0.20 |
| Vitamin E acetate | 0.30 |
| Propylene glycol | 5.00 |
| Perfume | q.s. |
| Preservative | q.s. |
| Ascorbic acid | 0.20 |
| Retinol 15D ® | 0.20 |
| Tocopherol | 0.10 |
| Water | ad 100 |

### EXAMPLE 2

Hand cream (W/O emulsion)

| | % w/w |
|---|---|
| Cetearyl alcohol | 1.00 |
| Glyceryl stearate | 1.50 |
| Stearyl alcohol | 1.50 |
| Cetyl palmitate | 2.00 |
| Vitamin E acetate | 0.50 |
| Dimethicone | 8.00 |
| Ceteareth-6 and stearyl alcohol | 3.00 |
| Octyl methoxycinnamate | 5.00 |
| Propylene glycol | 8.00 |
| Panthenol | 1.00 |
| Evening primrose oil | 3.00 |
| PEG-7 hydrogenated castor oil | 6.00 |
| Glyceryl oleate | 1.00 |
| Phenethyl dimethicone | 3.00 |
| Beeswax | 1.50 |
| Carob seed grain | 0.80 |
| Silk powder | 0.80 |
| Preservative | q.s. |
| Perfume | q.s. |
| Borax | 0.10 |
| Sodium ascorbate | 0.30 |
| Tocopherol | 0.60 |
| Retinol 15D ® | 0.66 |
| Water | ad 100 |

### EXAMPLE 3

Sunscreen lotion (W/O emulsion)

| | % w/w |
|---|---|
| PEG-7 hydrogenated castor oil | 6.00 |
| PEG-40 hydrogenated castor oil | 0.50 |
| Isopropyl palmitate | 7.00 |
| PEG-45/dodecyl glycol coplymer | 2.00 |
| Jojoba oil | 3.00 |
| Magnesium stearate | 0.60 |
| Octyl methoxycinnamate | 8.00 |
| C 12-15 alkyl benzoate | 5.00 |
| Titanium dioxide | 4.00 |
| Propylene glycol | 5.00 |
| EDTA | 0.20 |
| Preservative | q.s. |
| Retinol 15D ® | 0.33 |
| Water | ad 100 |
| Sodium ascorbyl phosphate | 1.00 |
| Vitamin E acetate | 0.50 |
| Sodium ascorbate | 0.20 |
| Tocopherol | 1.00 |
| Perfume | q.s. |

### EXAMPLE 4

Multiple emulsion (W/O/W emulsion)

| | % w/w |
|---|---|
| Paraffin oil | 7.50 |
| Cetearyl octanoate | 2.50 |
| Aluminum stearate | 0.25 |
| Magnesium stearate | 0.25 |
| Microcrystalline wax H | 0.50 |
| Cetearyl alcohol | 1.00 |
| Lanolin alcohol | 1.50 |
| Woolwax alcohol ointment | 1.50 |
| PEG-7 hydrogenated castor oil | 0.75 |
| PEG-45/dodecyl glycol copolymer | 2.00 |
| Ceteareth-6 and stearyl alcohol | 2.00 |
| Ceteareth-25 | 2.00 |
| Trilaureth-4 phosphate | 1.00 |
| Hydroxyethylcellulose | 0.20 |
| Propylene glycol | 7.50 |
| Magnesium sulfate | 0.25 |
| Ascorbic acid | 0.30 |
| Tocopherol | 0.01 |
| Retinol 15D ® | 0.40 |
| Water | ad 100 |

### EXAMPLE 5

Microemulsion

| | % w/w |
|---|---|
| Ceteareth-25 | 13.00 |
| PEG-7 glyceryl cocoate | 20.00 |
| Octyldodecanol | 5.00 |
| Preservative | q.s. |
| Ascorbic acid | 0.10 |
| Tocopherol | 0.10 |
| Retinol 15D ® | 0.66 |
| Water | ad 100 |

### EXAMPLE 6

Liposome gel (hydrophilic gel)

| | % w/w |
|---|---|
| PEG-40 hydrogenated castor oil | 1.00 |
| Bisabolol rac. | 0.10 |
| Propylene glycol | 8.00 |
| Panthenol | 0.50 |
| Water, vitamin E acetate, | 3.00 |
| polysorbate 80 and caprylic/capric | |
| triglyceride and lecithin | |
| Preservative | q.s. |
| Perfume | q.s. |
| Carbomer | 0.50 |
| Ascorbic acid | 0.15 |
| Tocopherol | 0.15 |
| Triethanolamine | 0.70 |
| Retinol 15D ® | 0.33 |
| Water | ad 100 |

### EXAMPLE 7

Cooling body splash (aqueous cosmetics)

| | % w/w |
|---|---|
| PEG-40 hydrogenated castor oil | 2.00 |
| Menthyl lactate | 0.20 |
| Alcohol | 5.00 |
| PEG-7 glyceryl cocoate | 2.00 |
| Hamamelis | 5.00 |
| Allantoin | 0.10 |
| Bisabolol rac. | 0.20 |
| Propylene glycol | 5.00 |
| Panthenol USP | 0.50 |
| Lactic acid (80% strength) | 0.20 |
| Ascorbic acid | 0.50 |
| Tocopherol | 0.30 |
| Perfume | q.s. |
| Retinol 15D ® | 0.20 |
| Water | ad 100 |

### EXAMPLE 8

Make-up (decorative cosmetics)

| | % w/w |
|---|---|
| Ceteareth-6 and stearyl alcohol | 9.00 |
| Dimethicone | 5.00 |
| Cetearyl octanoate | 8.00 |
| Macadamia nut oil | 5.00 |
| Propylene glycol | 5.00 |
| Retinol 15D ® | 0.66 |
| Water | ad 100 |
| Sicovit White E 171 | 8.00 |
| Sicomet Brown 70 13E 3717 | 2.00 |
| Ascorbic acid | 0.20 |
| Tocopherol | 0.50 |
| Perfume | q.s. |
| Benzophenone-3 | 5.00 |

### EXAMPLE 9

Fluid make-up (decorative cosmetics)

| | % w/w |
|---|---|
| Ceteareth-6 and stearyl alcohol | 7.00 |
| Ceteareth-25 | 5.00 |
| Dimethicone | 5.00 |
| Cetearyl octanoate | 8.00 |
| Macadamia nut oil | 5.00 |
| Propylene glycol | 5.00 |
| Retinol 15D ® | 0.33 |
| Water | ad 100 |
| Sicovit White E 171 | 8.00 |
| Sicomet Brown 70 13E 3717 | 1.00 |
| Ascorbic acid | 0.10 |
| Tocopherol | 0.01 |
| Perfume | q.s. |
| Benzophenone-3 | 5.00 |

1. A preparation comprising at least one retinoid, at least one water-soluble antioxidant and at least one oil-soluble antioxidant, where, per part by weight of retinoid, at least 1 part by weight of one or more water-soluble antioxidants and 0.1 to 100 parts by weight of one or more oil-soluble antioxidants are present in the preparation, where the content of one or more water-soluble antioxidants is in the range from 0.05 to 0.8% by weight, based on the total amount of the preparation.

2. A preparation as claimed in claim 1, which is a cosmetic or pharmaceutical preparation or food preparation.

3. A preparation as claimed in claim 1, comprising, per part by weight of retinoid, 1 to 5 parts by weight of one or more water-soluble antioxidants and 5 to 10 parts by weight of one or more oil-soluble antioxidants.

4. A preparation as claimed in claim 1, comprising, per part by weight of retinoid, 2 to 3 parts by weight of one or more water-soluble antioxidants and 8 to 10 parts by weight of one or more oil-soluble antioxidants.

5. A preparation as claimed in claim 1, comprising, as water-soluble antioxidant, L-ascorbic acid or salts of L-ascorbic acid.

6. A preparation as claimed in claim 1, comprising, as oil-soluble antioxidant, α-tocopherol.

7. A preparation as claimed in claim 1, wherein the retinoid is all-trans-retinol.

8. A preparation as claimed in claim 7, comprising 0.005 to 0.5% by weight of all-trans-retinol.

9. A preparation as claimed in claim 1 in the form of an O/W, W/O or multiple emulsion.

10. A preparation as claimed in claim 1, which is stored in oxygen-impermeable packagings without the addition of protective gas.

11. A preparation as claimed in claim 10, wherein the packaging is an aluminum tube.

12. A preparation as claimed in claim 11, wherein the inside of the aluminum tube is provided with a protective coating.

13. A preparation as claimed in claim 1, in the form of a skincare preparation.
